(19) United States
(12) Patent Application Publication
(10) Pub. No.: US 2008/0175885 A1
(43) Pub. Date: Jul. 24, 2008

(54) POROUS, DEGRADABLE IMPLANT MADE BY POWDER MOLDING

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/016,519

(22) Filed: Jan. 18, 2008

Related U.S. Application Data
(60) Provisional application No. 60/885,697, filed on Jan. 19, 2007.

(51) Int. Cl. A61F 2/02; B22F 3/11

(52) U.S. Cl. 424/426; 419/2

(57) ABSTRACT

The exemplary embodiment of the present invention are provided which relate to at least partially degradable implants and methods for the manufacture thereof which can use powder molding techniques. For example, a suspension can be provided comprising a plurality of first particles of at least one organic polymer, a plurality of second particles of at least one metal-based material which is at least partially biodegradable in-vivo, and at least one solvent. The first and second particles can be substantially insoluble in the solvent. The suspension can be molded to form a green body comprising the first particles embedded in a matrix of compressed second particles. The first particles may be removed from the green body by thermally induced decomposition and/or evaporation. Further, the green body can be sintered to form the implant. The first particles may be removed during the sintering procedure.

### CROSS-REFERENCE TO RELATED APPLICATIONS

The present invention claims priority of U.S. provisional application Ser. No. 60/885,697 filed Jan. 19, 2007, the entire disclosure of which is incorporated herein by reference.

### FIELD OF THE INVENTION

The present invention is directed to at least partially degradable implants and methods for the manufacture thereof which use powder molding techniques.

### BACKGROUND OF THE INVENTION

Implants are widely used as short-term or long-term devices to be implanted into the human body in different fields of application such as orthopedic, cardiovascular or surgical reconstructive treatments. Typically, implants are made of solid materials, either polymers, ceramics or metals. To provide improvements of engraftment or ingrowth of the surrounding tissue or adhesion, or to enable drug-delivery, implants have also been produced with porous structures. Different methods have been established to obtain either completely porous implants, particularly in the orthopedic field of application, or implants having at least porous surfaces, wherein a drug may be included for in-vivo release.

Powder metallurgy and powder shaping methods have been used for producing implants. For example, U.S. Pat. No. 7,094,371 describes a process for manufacturing porous artificial bone graft made of bioceramics such as hydroxyl apatite by extrusion molding of a slurry comprising ceramic powder, a gas-evolving pore-forming system and an organic binder. U.S. Publication No. 2006/0239851 and U.S. Publication No. 2006/0242813 A1 describe metal or powder injection molding processes for the production of metallic or ceramic parts or implants from injectable mixtures comprising a powder and thermoplastic organic binders such as waxes and polyolefins. These powder injection molding (PIM) or metal injection molding (MIM) processes include the sequential steps of injection molding a more or less net-shaped green part from the partially molten powder/binder mixture, substantially removing the binder to form a brown part, and subsequently sintering the brown part at high temperatures to produce the final product. Porosity may be created in these methods by adding placeholders such as inorganic salts or polymers which have to be removed before sintering.

The metal or ceramic powders used in these conventional PIM or MIM processes typically have particle sizes in the micrometer range, usually from 1 to 300 micrometer. After molding and removal of the binder, the parts made of such micro particles have to be sintered to form a mechanically stable product. Sintering is typically done at a temperature slightly below or close to the melting point of the material and held for a predetermined time, so that the particles may form bonds between each other and the material is densified.

German patent application DE 196 38 927 A1 describes a method for the manufacture of highly porous-shaped bodies by molding green bodies from mixtures of a metal powder and a placeholder material based on carbamide or melamine resin particles, followed by sublimation of the placeholder and subsequent sintering of the metal. The placeholder may be wetted by inert solvents and the mixture used for molding is a particulate agglomerate. Such essentially dry mixtures are typically not suitable for injection or extrusion molding, since extrusion molding conditions could lead to grinding and/or melting of the particulate agglomerates.

There may be an increasing need for porous materials to provide implant functionality with additional properties for drug-release or enhanced biocompatibility or the like. The requirements for such implants are increasingly complex, because the material properties must meet the mechanical requirements on the one hand, on the other hand the provision of functions such as drug-release requires a significant drug amount to be released and bio-available. Therefore, a sufficient compartment or space volume for desorption or deposition of the drug itself should preferably be provided without affecting the constructive properties of an implant, particularly its physical properties.

In addition, there may be a need for porous metal-based implants, whereas the pore size, the pore distribution and the degree of porosity can be adjusted without essentially deteriorating the physical and chemical properties of the material. Typically, with increasing degree of porosity the mechanical properties such as hardness and strength decrease over-proportionally. This is particularly disadvantageous in biomedical implants, where anisotropic pore distribution, large pore sizes and a high degree of porosity are required, whereas simultaneously a high long-term stability with regard to biomechanical stresses is necessary.

There may additionally be a need for providing drug-release function and improving the availability of the drug by increasing the overall volume of the compartment that contains the drug without adversely affecting the design of the device. For example, the conventional design of drug-eluting stents is based on non-porous scaffolds that have to be coated resulting in an increase of the stent strut thickness. By increasing the thickness results in adverse properties, such as increasing the profile of the stents within the target vessels, which can limit the use to large vessels, or which can be correlated to mechanically induced, haemodynamic-related thrombosis

Furthermore, there may be a need for drug-eluting implants which after implantation need to remain permanently in the body to fulfill, e.g., a permanent supporting function.

### SUMMARY OF EXEMPLARY EMBODIMENTS OF THE INVENTION

It is one object of the present invention to provide a temporary implant capable of releasing active ingredients such as e.g. a drug or a marker etc. Another object of the invention is to provide implants with sufficient pore volume, whereby the pore sizes are controllable for incorporating large amounts of active ingredients.

Exemplary embodiments of manufacturing methods should include possibilities to accurately control pore sizes, mechanical and dimensional properties, chemical and physical properties, as well as simplifying the manufacturing process and reducing manufacturing costs.

According to one aspect the present invention, a method can be provided for the manufacture of an at least partially biodegradable, porous implant or a part thereof. For example, a suspension can be provided comprising a plurality of first particles of at least one organic polymer; a plurality of second particles of at least one metal-based material which is at least partially biodegradable in-vivo; and at least one solvent. The first and second particles may be substantially insoluble in the solvent. The suspension may be molded to form a green body comprising the first particles embedded in a matrix of compressed second particles. The first particles can be removed from the green body by thermally induced decomposition and/or evaporation. Further, the green body can be sintered to form the implant. The first particles can be removed during the sintering.

Unlike conventional methods which essentially require removal of the binder and other materials in a separate step before the step of sintering at high temperatures, or at least a temperature plateau during sintering, the embodiments of the present invention use a one-step procedure, wherein the first particles are decomposed essentially during sintering. This may be carried out, e.g. by essentially rapidly and/or continuously heating the shaped body to the sintering temperature, without prior thermal treatment steps

Unlike conventional methods which essentially require removal of the binder and other materials in a separate step before the step of sintering at high temperatures, or at least a temperature plateau during sintering, the exemplary embodiments of the present invention can generally employ a single-step procedure, whereas the first particles may be decomposed essentially during sintering. This may be done, e.g., by essentially rapidly and/or continuously heating the shaped body to the sintering temperature, without prior thermal treatment (other than drying) or plateaus in the heating ramp, i.e. holding the temperature constant at a level between drying temperature and the final sintering temperature for extended periods of more than e.g. 5 minutes.

Suitable heating ramps can be, e.g., from about 0.1 K/min up to 40 K/min, such as from about 5 K/min up to 20 K/min, or from about 15 to 25 K/min, or from about 7 K/min up to 10 K/min, most preferably at about 20 K/min. According to still another exemplary embodiment of the present invention, the heating ramps can be continuously applied, without interruption or plateaus in the temperature profile up to reaching the final sintering temperature. One of the advantages of rapid heating is—without referring to any specific theory—that the sintering process itself can take place without significantly altering the pore shape and volume created by the thermally degradable particles. A two-step approach with first partially removing the thermally degradable material before the final sintering step typically results in melting of the organic polymer and a decrease of the viscosity of the mixture, leading to a collapse of the larger pores. These effects may cause a destruction of the fine-structure and arrangement of the particles that shall be sintered without significantly affecting the shape and size of the removable particles.

In further exemplary embodiments of the present invention, the suspension can be molded by compacting, injection molding, uniaxial or biaxial pressing, isostatic pressing, slip casting, and/or extrusion molding procedure(s). The injection molding or extrusion molding procedures may be preferred options, for example, from flowable, paste-like suspensions.

The first and second particles may be independently selected from spherical particles, dendritic particles, cubes, wires, fibers and/or tubes, and the metal-based particles can include a metal, a metal alloy, a metal oxide, a metal carbide, a metal nitride and/or a metal-containing semiconductor.

Typical examples for biodegradable metal-based particles can include Mg or Zn, or an alloy comprising at least one of Mg, Ca, Fe, Zn, Al, W, Ln, Si, or Y.

In a further exemplary embodiment of the present invention, at least partially biodegradable porous implant can be provided which is producible by the method as described above. The implant exemplary may include an active ingredient, such as a biologically or pharmacologically active agent, a diagnostically active agent, or a combination of both, and the implant may be one of a vascular endoprosthesis, an intraluminal endoprosthesis, a stent, a stent graft, a coronary stent, a peripheral stent, a surgical or orthopedic implant, an implantable orthopedic fixation aid, an orthopedic bone prosthesis or joint prosthesis, a bone substitute or a vertebral substitute in the thoracic or lumbar region of the spinal column; an artificial heart or a part thereof, an artificial heart valve, a heart pacemaker casing or electrode, a subcutaneous and/or intramuscular implant, an implantable drug-delivery device, a microchip, or implantable surgical needles, screws, nails, clips and/or staples. Optionally, the exemplary implant may be active agent-eluting, e.g., configured to release at least one active ingredient in-vivo or ex-vivo.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a schematical illustration of, at the left hand side thereof, a tubular implant according to an exemplary embodiment or the present invention, and a partial magnification of the structure thereof illustrating the structure that is composed of or manufactured from a plurality of spherical particles surrounding larger voids left over from removed particles; and

FIG. 2 is a schematic illustration of a three-dimensional orientation of the spherical particles surrounding larger voids left over from removed particles.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

The term “biodegradable” as used herein can include but not limited to any material which can be removed in-vivo, e.g. by biocorrosion or biodegradation. Thus, any material, e.g., a metal or organic polymer that can be degraded, absorbed, metabolized, or which is resorbable in the human or animal body may be used either for a biodegradable metallic layer or as a biodegradable template in the embodiments of the present invention. In addition, as used in this description, the terms “biodegradable”, “bioabsorbable”, “resorbable”, and “biocorrodible” can include, and not limited to encompassing materials that are broken down and may be gradually absorbed or eliminated by the body in-vivo, regardless of whether these processes are due to hydrolysis, metabolic processes, bulk or surface erosion.

The terms “active ingredient”, “active agent” or “beneficial agent” as used herein can include but not limited to any material or substance which may be used to add a function to the implantable medical device. Examples of such active ingredients can include biologically, therapeutically or pharmacologically active agents such as drugs or medicaments, diagnostic agents such as markers, or absorptive agents. The active ingredients may be a part of the first or second particles, such as incorporated into the implant or being coated on at least a part of the implant. Biologically or therapeutically active agents comprise substances being capable of providing a direct or indirect therapeutic, physiological and/or pharmacological effect in a human or animal organism. A therapeutically active agent may include a drug, pro-drug or even a targeting group or a drug comprising a targeting group. A term “active ingredient” according to the exemplary embodiments of the present invention may further include a material or substance which may be activated physically, e.g. by radiation, or chemically, e.g. by metabolic processes.

Without wishing to be bound to any particular theory, by molding suspensions of polymeric particles and metal-based particles under sufficiently high pressures, mechanically stable porous implantable devices may be produced, which can be easily functionalized, for example, for the eluting of drugs or for improving the visibility of the implant in the body. The use of nanoparticles as the metal-based particles instead of conventionally used micro particles can provide sufficient mechanical stability, so that after sintering, highly porous implants may be obtained in complex geometries which have sufficient mechanical stability to be used, even under high strains. By the methods as described herein, porous implants may be produced in any desired shape by compacting and sintering flowable suspensions of polymeric particles and metal-based particles to produce the implants in a substantial net-shape. A wide variety of compaction molding procedures may be used.

Metal-Based Particles

According to the exemplary embodiments of the present invention, the basic implant structure can be made from biodegradable metal-based particles, which after molding, form a matrix into which the organic polymer particles are temporarily embedded as place-holders. The organic polymer particles are removed during sintering and their size, amount and distribution in the metal-based particle matrix essentially determine the interior structure and porosity of the implant. The biodegradable metal-based particles may be selected from at least partially biodegradable inorganic materials such as metals or ceramics or any mixture thereof to provide the structural body of the implant. The term biodegradable as used herein can include any material which can be removed in-vivo, e.g. by biocorrosion or biodegradation. Thus, any metal-based particle that can be degraded, absorbed, metabolized, is resorbable in the human or animal body may be used as the biodegradable metal-based particle in the embodiments of the present invention.

According to one exemplary embodiment of the present invention, the porous implant can be made from biodegradable metal-based particles, which may be selected from any suitable bio-corrodible material to provide the structural body of the implant. The metal-based particles can include, e.g., metals, metal compounds such as metal oxides, carbides, nitrides and mixed forms thereof, or metal alloys, e.g. particles or alloyed particles including alkaline or alkaline earth metals, Fe, Zn or Al, such as Mg, Fe or Zn, and optionally alloyed with or combined with other particles selected from Mn, Co, Ni, Cr, Cu, Cd, Pb, Sn, Th, Zr, Ag, Au, Pd, Pt, Si, Ca, Li, Al, Zn and/or Fe. Also suitable are, e.g., alkaline earth metal oxides or hydroxides such as magnesium oxide, magnesium hydroxide, calcium oxide, and calcium hydroxide or mixtures thereof. In exemplary embodiments, the biodegradable metal-based particles may be selected from biodegradable or biocorrosive metals or alloys based on at least one of magnesium or zinc, or an alloy comprising at least one of Mg, Ca, Fe, Zn, Al, W, Ln, Si, or Y. Furthermore, the implant may be substantially completely or at least partially degradable in-vivo. Examples for suitable biodegradable alloys comprise e.g. magnesium alloys comprising more than 90% of Mg, about 4-5% of Y, and about 1.5-4% of other rare earth metals such as neodymium and optionally minor amounts of Zr; or biocorrosive alloys comprising as a major component tungsten, rhenium, osmium or molybdenum, for example alloyed with cerium, an actinide, iron, tantalum, platinum, gold, gadolinium, yttrium or scandium.

The exemplary metal particles, alloy particles or particle mixtures may include, in an exemplary embodiment:
(i) 10-98 wt.-%, such as 35-75 wt.-% of Mg, and 0-70 wt.-%, such as 30-40% of Li and 0-12 wt.-% of other metals, or
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals, wherein the individual weight ranges are selected to always add up to 100 wt.-% in total for each alloy.

In certain exemplary embodiments of the present invention, the implant can be mainly degraded to hydroxyl apatite within the living body. This property of the inventive implant material can be especially advantageous for joint implants, bone implants and grafts, nails, screws and the like.

The metal-based particles can be used in the form of powders, which are, for example, obtainable by conventional methods such as electrochemical or electrolytic methods, spraying methods such as a rotating electrode process which can lead to spherical particles, or chemical gas phase reduction, flame pyrolysis, plasma methods, high energy milling or precipitation methods.

In the exemplary embodiments of the present invention, the metal-based particles can have a form as desired, for example selected from spherical particles, dendritic particles, cubes, wires, fibers or tubes.

In further exemplary embodiments of the present invention, the metal-based particles of the above mentioned materials can include nano- or microcrystalline particles, nanofibers or nanowires. Without wishing to be bound to any particular theory, ultra fine nano-sized particles or nanoparticles as the metal-based particles are particularly useful for manufacturing the implants of the exemplary embodiments of the present invention. In further exemplary embodiments, it can be preferred to be selected from nano-alloys.

The metal-based particles useful according to ain exemplary embodiments of the present invention can have an average (D50) particle size from about 0.5 nm to 500 μm, preferably below about 1,000 nm, such as from about 0.5 nm to 1,000 nm, or below 900 nm, such as from about 0.5 nm to 900 nm, or from about 0.7 nm to 800 nm.

Preferred D50 particle size distributions can be in a range of about 10 nm up to 1,000 nm, such as between 25 nm and 600 nm or even between 30 nm and 250 nm. Particle sizes and particle distribution of nano-sized particles may be determined by spectroscopic methods such as photo correlation spectroscopy, or by light scattering or laser diffraction techniques.

The metal-based compounds can be encapsulated in particles or coated on polymer particles in the process of certain exemplary embodiments of the present invention. The metal-based particles can also comprise mixtures of different metal-based particles, particularly having different specifications, e.g. the corrosion rate in physiological fluids, or chemical and/or physical properties, such as absorption of x-ray or ferromagnetic properties, in accordance with the desired properties of the implant to be produced. The metal-based particles may be used in the form of powders, sols, colloidal particles, dispersions, or suspensions.

In further exemplary embodiments, particularly for implants with magnetic or signaling properties in general, magnetic metals or alloys such as ferrites, e.g. gamma-iron oxide, magnetite or ferrites of Co, Ni, Mn can be selected as at least a part of the metal-based particles used. Materials having signaling properties are those materials which, when implanted into the human or animal body, can produce a signal which is detectable by imaging methods such as x-ray, nuclear magnetic resonance, szintigraphy, etc.

For example, to improve the imaging properties of the biodegradable material, semiconducting nanoparticles can be used as a part of the metal-based particles in some embodiments, such as e.g. semiconductors of groups II-VI, groups III-V, or group IV of the periodic system. Suitable group II-VI-semiconductors are, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, or mixtures thereof. Examples for group III-V semiconductors are GaAs, GaN, GaP, GaSb, InGaAs, InP, InN, InSb, InAs, AlAs, AlP, AlSb, AlS, or mixtures thereof. Examples for group IV semiconductors are germanium, lead and silicon. The semiconductors may also be used in the form of core-shell-particles. Also, combinations of any of the foregoing semiconductors may be used. Also, complex formed metal-based nanoparticles may be used to replace some of the biodegradable metal-based particles, for example so-called core-shell configurations, as described explicitly by Peng et al., “Epitaxial Growth of Highly Luminescent CdSe/CdS Core/Shell Nanoparticles with Photo stability and Electronic Accessibility”, Journal of the American Chemical Society, (1997) 119:7019-7029. Preferred in certain exemplary embodiments can be semiconducting nano-particles selected from those as listed above, having a core with a diameter of about 1 to 30 nm, such as from about 1 to 15 nm, upon which further semiconducting nano-particles in about 1 to 50 monolayers, such as about 1 to 15 monolayers are crystallized as a shell. Core and shell may be present in nearly any combination of the materials as described above, preferred in some exemplary embodiments are CdSe and CdTe as core and CdS and ZnS as in the shell in such particles.

In a further exemplary embodiment of the present invention, the metal-based particles can be selected due to their absorptive properties for radiation in a wavelength range from gamma radiation up to microwave radiation, or due to their property to emit radiation, particularly in the region of 60 nm or less. By suitably selecting the metal-based particles, the inventive process can lead to the production of implants having non-linear optical properties, for example materials that block IR-radiation of specific wavelengths, suitable for marking purposes or for therapeutic implants absorbing radiation, which may be used e.g. in cancer therapy.

In certain exemplary embodiments of the present invention, to improve imaging properties of the implant material, at least a part of the metal-based particles, their particle sizes and their diameter of core and shell can be selected from photon-emitting compounds, such that the emission is in the range from 20 nm to 1000 nm, or from a mixture of suitable particles which emit photons of differing wavelengths when exposed to radiation. In an exemplary embodiment, fluorescent metal-based particles are selected which need not to be quenched.

Organic Polymer Particles

To create porosity in the implants of the exemplary embodiments of the present invention, pore-forming organic polymer particles can be embedded in the metal-based particles during molding, which are subsequently removed during sintering. The free space left by the removed polymer particles can essentially define the pores, their number and size and thus the overall porosity of the implant. For example, the polymer particles can serve as place-holders or templates for a hollow space or pore during molding of the green body, which define the porous compartments or sections in shape and size of free space created after removal of the polymer particles. The organic polymer particles to be embedded in the metal-based particles may have any desired form such as spherical, cubic, dendritic or fibrous particles or any mixture thereof.

In the certain exemplary embodiments of the present invention, the pore-forming organic polymer particles can be thermally degradable, vaporizable, i.e. they may be substantially completely decomposed under the conditions of elevated temperatures during sintering.

Polymers which may be used for the polymer particles include, for example, poly(meth)acrylate, unsaturated polyester, saturated polyester, polyolefines such as polyethylene, polypropylene, polybutylene, alkyd resins, epoxy-polymers or resins, polyamide, polyimide, polyetherimide, polyamideimide, polyesterimide, polyester amide imide, polyurethane, polycarbonate, polystyrene, polyphenol, polyvinyl ester, polysilicone, polyacetal, cellulosic acetate, polyvinyl chloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, cyanatoester-polymers, and mixtures or copolymers of any of the foregoing are preferred polymeric particles.

In certain exemplary embodiments of the present invention, the pore-forming polymer particles can be selected from poly(meth)acrylates based on mono(meth)acrylate, di(meth)acrylate, tri(meth)acrylate, tetra-acrylate and pentaacrylate; as well as mixtures, copolymers and combinations of any of the foregoing.

Without referring to a specific theory, the shape and the size of the pore-forming polymer particles can result in a reproducible and rationally designable final structure of the sintered implant body. For example, using fibrous polymer particles can provide fibrous cavities or hollow compartments or sections within the sintered implant, and the use of spherical particles typically provides essentially spherical cavities, whereby mixing both particle types entities can result in the formation of both fibrous and spherical cavities, e.g. porous compartment or sections of a more complex geometry.

Molding

To mold the particles into a desired shape, a suspension of the particles can be formed. In exemplary embodiments of the present invention, the metal-based particles and the organic polymer particles can be suspended in a suitable solvent, to form a suspension or a paste, i.e. a dispersion of both types of particle in a liquid, flowable medium. Thus, the solvent should be inert, i.e. it has to be selected such that the metal-based particles and the polymer particles are substantially insoluble in the solvent, and the solvent should not degrade the biocorrosive metal-based particles.

Moldable suspensions can include, depending on the particles selected, solvents such as alcohols, ethers, hydrocarbons or water. Examples may include methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofurane, trimethyl hexanol, phenol, benzene, toluene, xylene; as well as water, if necessary mixed with dispersants, surfactants or other additives and mixtures of the above-named substances. In some embodiments, it is suitable to use liquid nitrogen or carbon dioxide as a solvent.

Further, a wetting agent can be added to the metal-based particles or to the moldable suspension, e.g. Byk P-104 (BYK-Chemie, Germany), to improve dispersibility of the nano-sized particles.

The moldable suspension can have at minimum 50% by weight solids content of the metal-based particles, such as about 60 to 80 wt.-%, and not more than 40 wt.-% of the solids content of the polymer particles. The solvent content in the suspension typically does not exceed 50 wt.-% of the moldable composition, such as 30 wt.-% or less than 10 wt.-%. The suspension can be viscous, such as paste-like. Typical viscosities (at 20° C.) of the moldable suspension may be above about 10³ mPa·s, e.g. at about 10³ to 10¹⁰ mPa·s, such as about 10³ to 10⁶ mPa·s, or at about 10⁴ to 10⁵ mPa·s.

Preparation of the suspension can be performed by applying conventional processes to obtain substantially homogeneous suspensions. In some embodiments, it can be preferred not to use any solvent, but to mix the particles based on dry methods and to mold the implant from a substantially dry powder mixture.

A variety of conventional molding techniques can be used in the exemplary embodiments of the present invention for molding the implant. Such molding techniques can include, for example, injection molding, compression molding, compacting, dry pressing, cold isostatic pressing, hot pressing, uniaxial or biaxial pressing, extrusion molding, gel casting, slip casting and/or tape casting.

A suitable compacting device that achieves uniform compacting forces can be a floating mold die press. The compaction pressure may determine the density of the molded green body and the final implant. If the compaction pressure is too low, the green body and the implant can have a lower than desired density and not attain the desired net shape. The molded green body or the final implant can delaminate and result in a material that is defective for the intended use if the compaction pressure is too high. The compaction pressure suitable in the embodiments of the present invention can be in the range of from about 1,000 psi (6.89 MPa) to 20,000 psi (138 MPa), such as from about 5,000 psi to 15,000 psi, or about 10,000 psi (68.9 MPa).

The compaction time can be readily determined by the operator depending on the compaction pressure selected. Compaction time, for example, can be in the range of from about 60 seconds to 10 seconds for compaction pressures in the range of from 10,000 psi to 15,000 psi, respectively, and 30 seconds for a compaction pressure of 12,000 psi. For example, to produce a near-net shape implant according to the invention, i.e. an implant which is dimensionally almost identical to the molded green body, the compacting is carried out for a time sufficient to compact the precursor to form a molded implant having a predetermined density, for example, from about 1.0 g/cc to 10.5 g/cc. The compaction pressure and time selected by the operator can be dependent on the size of the finished part. Generally, as the part size increases, compaction pressure and/or compaction time increase.

Another exemplary embodiments of the present invention can include the preference for the mechanical stability of the final implant. For example, for stents, it may be desirable to have a higher density of the particles and a more compact implant body to allow sufficient electromechanically stability for crimping on balloon catheters and subsequent expansion during the intended use.

The molds can be selected as desired, suitable for the specific design of any implant. The implantable medical devices to be selected are not limited to any particular implant type, so that, for example, however not exclusively, the implant producible by the exemplary embodiments of the method according to the present invention can include vessel endoprostheses, intraluminal endoprostheses, stents, coronary stents, peripheral stents, pacemakers or parts thereof, surgical and orthopedic implants for temporary purposes, such as joint socket inserts, surgical screws, plates, nails, implantable orthopedic supporting aids, surgical and orthopedic implants, such as bones or joint prostheses, for example artificial hip or knee joints, bone and body vertebra means, artificial hearts or parts thereof, artificial heart valves, cardiac pacemaker housings, electrodes, subcutaneous and/or intramuscular implants, active substance repositories or microchips or the like, also injection needles, tubes and/or endoscope parts.

With the process of the exemplary embodiments of the present invention, implants may be manufactured, e.g., in one seamless part or with seams from multiple parts. The implants or parts thereof, such as semifinished parts, may be manufactured in the desired shape using conventional implant manufacturing techniques. For example, suitable manufacturing methods may include, but are not limited to, laser cutting, chemical etching, stamping of tubes, or stamping of flat sheets, rolling of the sheets and, as a further option, welding or gluing the sheets, e.g. to form tubular stents. Other manufacturing techniques include electrode discharge machining or molding the inventive implant with the desired design. A further option may be to weld or glue individual sections of the implant together.

Pore Design

Without referring to a specific theory, the shape and the size of the degradable polymer particles can result in a reproducible and rationally designable structure of the implant after decomposition or removal of the polymer particles. For example, using fibrous polymer particles can result in the forming of fibrous cavities, or using cubic particles can result in forming cubic cavities within the implants. Using spherical particles can result in spherical cavities, whereby mixing of different particle types entities results in combinations or more complex formations of fibrous and spherical cavities, e.g. open porous networks.

The design of pores, pore sizes, shapes and pore volume, depends on the implant and its intended use as well as implant function. A person of ordinary skill in the art can easily determine the amount of organic polymer particles preferable to obtain a specific volume of pores left in the implant after removal of the polymer. Pore volumes can be increased either by using larger-sized polymer particles or increasing the total amount of smaller-sized polymer particles. Depending on the intended use and functional requirements in some exemplary embodiments, it may also be beneficial to adjust the size of the metal-based particles in order to obtain a suitable grain size of the implant and to increase the structural integrity. The selection of the size of polymer particles can also determine the resulting size of the pores within the implant. For the polymer particles, spherical particles may be selected with a size from about 2 nm up to 5,000 μm, such as from about 10 nm up to 1,000 nm or from about 100 nm up to 800 nm. In some embodiments, a structure of hierarchical porosities may be obtained by combining different sizes or shapes of polymer particles. In some embodiments, fibrous polymer particles may be used, e.g. having a thickness of about 1 nm to 5,000 μm, such as from about 20 nm to 1,000 nm, or from about 50 nm to 600 μm. The length of fibrous particles can be at about 100 nm to 10,000 μm, such as from about 100 nm to 1,000 μm or from about 200 nm to 1,000 nm. In some exemplary embodiments, spherical and fibrous polymer particles may be combined.

A person of ordinary skill in the art can easily calculate the ratio of both particle types based on the densities of the metal-based particles and polymer particles. To increase the mechanical stability and structural integrity of the implant, the ratio of the particle sizes of both particle types may be adjusted. In certain exemplary embodiments, a D50 size ratio of metal-based particles versus polymer particles may be at about 1:1, or about 2:1, or about 5:1. In other embodiments, it can be more appropriate to use the particles in a ratio of about 1:2, or from about 1:5 or 1:20, or 1:30. Any other ratio may be suitable according to the exemplary embodiments of the present invention, depending on the final implant and the desired shape, function and mechanical properties.

Sintering

After molding the suspension into a green body comprising the polymer particles embedded in a matrix of the metal-based particles, a sintering process can be applied in the embodiments of the method of the exemplary embodiments of the present invention. Sintering is typically carried out at a temperature slightly below or close to the melting point of the material and held for a predetermined time period, so that the metal-based particles may form bonds between each other to improve the mechanical stability. Optionally and depending on the materials, the amount ratios thereof used and the molding conditions, the material may be densified upon sintering. In an exemplary embodiment of the present invention, the removal of the polymer particles occurs during or substantially simultaneous to sintering, respectively.

Sintering of nanoparticulate metal-based materials can allow for using lower temperatures compared to conventional metal welding or metal injection molding methods which typically use micron-sized particles. The temperatures for sintering and removal of the polymer particles can be in the range of 100° C. to 1500° C., preferably in the range of 300° C. to 800° C., and particularly in the range of 400° C. to 600° C.

During thermal treatment, the pore-forming polymer particles can be thermolytically degraded or decomposed. The structural integrity and homogeneity of the obtained porous metal or metal oxide implant can also depend on the selection of appropriate heating ramps and the duration time of the thermal process. The parameters can be selected by the operator according to the requirements for the final implant.

To obtain the final implant, a thermal treatment can be used to remove the polymer particles and to sinter the metal-based particles in an essentially one-step procedure that yields a sintered metal implant having a porous structure. Conventional methods typically use a two-step thermal treatment to remove, for example, an organic binder substantially completely at a relatively lower temperature than the actual sintering step requires, which is performed subsequently after significantly further raising the temperature. Such two-step procedures generally include methods where the green body is heated up with a first heat ramp to a first temperature (plateau temperature) held for a certain period of time to evaporate the place-holder or binder, and then raising the temperature with a second heat ramp to a second temperature to sinter the metals.

In the exemplary embodiments of the present invention, a single-step procedure for removal of organics and sintering can be used, e.g, a procedure using a single ramp for raising the temperature up to the sintering temperature, substantially with no plateaus in the temperature profile, as described above and with the heating ramps as described above. For example, a suitable heating ramp may be up to about 25 K/min, e.g. 20 K/min, 15 K/min, or in some embodiments even below about 7 K/min, such as below about 3 K/min.

Depending on the intended final implant material, the thermal treatment may be performed in an inert gas atmosphere, for example to avoid oxidation of the metal or to avoid contaminations. Suitable inert gases include, e.g. nitrogen, SF₆, noble gases like argon, helium or any mixtures thereof. Also, reactive atmospheres during sintering may be used, e.g. to facilitate decomposition of the polymer particles, for example oxidizing atmospheres comprising e.g. oxygen, carbon monoxide, carbon dioxide, or nitrogen oxide. Furthermore, it is possible to blend the inert atmosphere with reactive gases, e.g. hydrogen, ammonia, C₁-C₆ saturated aliphatic hydrocarbons such as methane, ethane, propane and butane, or mixtures thereof.

In certain exemplary embodiments of the present invention, it may be preferred that the atmosphere during the process is substantially free of oxygen. The oxygen content may be below about 10 ppm, or even below 1 ppm.

Functional Modification

Functional modification can be done, for example, by incorporating an active ingredient into the pores of the implant structure. The active ingredient may be configured to be released from the implant in-vivo or ex-vivo, e.g. to provide a drug eluting implant. In other exemplary embodiments, functional modification can involve coating the produced implant partially or completely with an active ingredient. Active ingredients may comprise therapeutically active agents such as drugs or medicaments, diagnostic agents such as markers, or absorptive agents. In further exemplary embodiments, the therapeutically active, diagnostic or absorptive agents can be part of the metal-based particles and thus a part of the implant body.

Therapeutically active agents suitable for being incorporated into the implant or for being coated on at least a part of the implant, according to exemplary embodiments of the present invention, may be preferably therapeutically active agents which are capable of providing direct or indirect therapeutic, physiological and/or pharmacological effect in a human or animal organism. In an alternative exemplary embodiment, the active ingredient may also be a compound for agricultural purposes, for example a fertilizer, pesticide, microbicide, herbicide, algaecide etc. The therapeutically active agent may be a drug, pro-drug or even a targeting group or a drug comprising a targeting group.

The active ingredients may be in crystalline, polymorphous or amorphous form or any combination thereof in order to be used in the exemplary embodiments of the present invention.

Suitable therapeutically active agents may be selected from the group of enzyme inhibitors, hormones, cytokines, growth factors, receptor ligands, antibodies, antigens, ion binding agents such as crown ethers and chelating compounds, substantial complementary nucleic acids, nucleic acid-binding proteins including transcriptions factors, toxins etc. Examples of such active agents are, for example, cytokines such as erythropoietine (EPO), thrombopoietine (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxins including ricine and further active agents such as those included in Physician's Desk Reference, 58th Edition, Medical Economics Data Production Company, Montvale, N.J., 2004 and the Merck Index, 13th Edition (particularly pages Ther-1 to Ther-29).

In an exemplary embodiment of the present invention, the therapeutically active agent can be selected from the group of drugs for the therapy of oncological diseases and cellular or tissue alterations. Suitable therapeutic agents are, e.g., antineoplastic agents, including alkylating agents such as alkyl sulfonates, e.g., busulfan, improsulfan, piposulfane, aziridines such as benzodepa, carboquone, meturedepa, uredepa; ethyleneimine and methylmelamines such as altretamine, triethylene melamine, triethylene phosphoramide, triethylene thiophosphoramide, trimethylolomelamine; so-called nitrogen mustards such as chlorambucil, chlomaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds such as carmustine, chlorozotocin, fotemustine, lomustine, nimustine, ranimustine; dacarbazine, mannomustine, mitobranitol, mitolactol; pipobroman; doxorubicin and cis-platinum and its derivatives, etc., combinations and/or derivatives of any of the foregoing.

In a further exemplary embodiment of the present invention, the therapeutically active agent is selected from the group of anti-viral and anti-bacterial agents such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolic acid, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, etc., combinations and/or derivatives of any of the foregoing.

In a further exemplary embodiment of the present invention, the therapeutically active agent may include a radio-sensitizer drug, or a steroidal or non-steroidal anti-inflammatory drug.

In a further exemplary embodiment of the present invention, the therapeutically active agent is selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 etc., combinations and/or derivatives of any of the foregoing.

In a further exemplary embodiment of the present invention, the therapeutically-active agent is selected from the group of nucleic acids, wherein the term nucleic acids also comprises oligonucleotides, wherein at least two nucleotides are covalently linked to each other, for example in order to provide gene therapeutic or antisense effects. Nucleic acids preferably comprise phosphodiester bonds, which also comprise those which are analogues having different backbones. Analogues may also contain backbones such as, for example, phosphoramide (Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996). Further analogues are those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook. The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36. Besides the selection of the nucleic acids and nucleic acid analogues known in the prior art, also a mixture of naturally occurring nucleic acids and nucleic acid analogues or mixtures of nucleic acid analogues may be used.

In a further embodiment of the present invention, the therapeutically active agent can be selected from the group of metal ion complexes, as described in International Application Nos. PCT/US95/16377, PCT/US96/19900, and PCT/US96/15527, whereas such agents reduce or inactivate the bioactivity of their target molecules, preferably proteins such as enzymes.

Therapeutically active agents may also include anti-migratory, anti-proliferative or immune-suppressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, statines and others, their derivatives and analogues.

Active agents or combinations of active agents may be further selected from heparin, synthetic heparin analogues (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid [aspirin], ticlopidine, clopidogrel, abciximab, dextrans; corticosteroids such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs) such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics such as alkaloides and podophyllum toxins such as vinblastine, vincristine; alkylating agents such as nitrosoureas, nitrogen lost analogues; cytotoxic antibiotics such as daunorubicin, doxorubicin and other anthracyclines and related substances, bleomycin, mitomycin; antimetabolites such as folic acid analogs, purine analogs or pyrimidine analogs; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrhythmics, in particular, class I antiarrhythmic such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenyloin, tocainid; class Ic antiarrhythmics, e.g., propafenon, flecainid(acetate); class II antiarrhythmics beta-receptor blockers such as metoprolol, esmolol, propranolol, metoprolol, atenolol, oxprenolol; class III antiarrhythmics such as amiodarone, sotalol; class IV antiarrhythmics such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers, such as prazosin, urapidil, doxazosin, bunazosin, terazosin, indoramin; vasodilatators such as dihydralazine, diisopropylamine dichloracetate, minoxidil, nitroprusside sodium; other antihypertensives such as indapamide, co-dergocrine mesylate, dihydroergotoxin methanessulfonate, cicletanin, bosentan, fludrocortisone; phosphodiesterase inhibitors such as milrinon, enoximon and antihypotensives, such as, in particular, adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGF, PDGF, VEGF, bFGF, TNF, NGF, GM-CSF, IGF-a, IL-1, IL 8, IL-6, growth hormone; as well as adhesive substances such as cyanoacrylates, beryllium, silica; and growth factors such as erythropoetin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particularly recombinant BMPs, such as recombinant human BMP-2 (rhBMP-2), bisphosphonate (e.g., risedronate, pamidronate, ibandronate, zoledronic acid, clodronic acid, etidronic acid, alendronic acid, tiludronic acid), fluorides, such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs, such as, in particular, β-lactam antibiotics, e.g., β-lactamase-sensitive penicillins such as benzyl penicillins (penicillin G), phenoxymethylpenicillin (penicillin V); β-lactamase-resistant penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactainase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscamet, inosine-(dimepranol-4-acetamidobenzoate), valganciclovir, valaciclovir, cidofovir, brivudin; antiretroviral active ingredients (nucleoside analogue reverse-transcriptase inhibitors and derivatives) such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In an alternative exemplary embodiment of the present invention, the active agents can be encapsulated in polymers, vesicles, liposomes or micelles.

Suitable diagnostically active agents for use in the exemplary embodiments of the present invention can be e.g. signal generating agents or materials, which may be used as markers. Such exemplary signal generating agents can include materials which in physical, chemical and/or biological measurement and verification methods lead to detectable signals, for example in image-producing methods. It is not important for the present invention whether the signal processing is carried out exclusively for diagnostic or therapeutic purposes. Typical imaging methods are, for example, radiographic methods, which are based on ionizing radiation, for example conventional X-ray methods and X-ray based split image methods such as computer tomography, neutron transmission tomography, radiofrequency magnetization such as magnetic resonance tomography, further by radionuclide-based methods such as scintigraphy, Single Photon Emission Computed Tomography (SPECT), Positron Emission Computed Tomography (PET), ultrasound-based methods or fluoroscopic methods or luminescence or fluorescence based methods such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Exemplary signal generating agents can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds.

Exemplary metal-based agents can be, e.g., nanomorphous nanoparticles from metals, metal oxides, semiconductors as defined above as the metal-based particles, or mixtures thereof. In this regard, it may be preferred to select at least a part of the metal-based particles from those materials capable of functioning as signal generating agents, for example to mark the implant for better visibility and localization in the body after implantation.

Further, exemplary signal producing metal-based agents can be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) etc. Based on especially pronounced magnetic moments, especially gadolinium (III), terbium (III), dysprosium (III), holmium (III) and erbium (III) are mostly preferred. Further one can select from radioisotopes. Examples of a few applicable radioisotopes include H 3, Be 10, O 15, Ca 49, Fe 60, In 111, Pb 210, Ra 220, Ra 224 and the like. Typically such ions are present as chelates or complexes, wherein, for example, as chelating agents or ligands, for lanthanides and paramagnetic ions compounds such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are, for example, published in Alexander, Chem. Rev. 95:273-342 (1995) and Jackels, Pharm. Med. Imag, Section III, Chap. 20, p645 (1990). Other usable chelating agents are described in U.S. Pat. Nos. 5,155,215; 5,087,440; 5,219,553; 5,188,816; 4,885,363; 5,358,704; 5,262,532, and Meyer et al., Invest. Radiol. 25: S53 (1990), U.S. Pat. Nos. 5,188,816, 5,358,704, 4,885,363 and 5,219,553. In addition, salts and chelates from the lanthanide group with the atomic numbers 57-83 or the transition metals with the atomic numbers 21-29, or 42 or 44 may be incorporated into the implants of exemplary embodiments of the present invention.

In addition, paramagnetic perfluoroalkyl-containing compounds can also be suitable, which, for example, are described in German Patent Nos. DE 196 03 033 and DE 197 29 013 and in International Patent Publication WO 97/26017; furthermore diamagnetic perfluoroalkyl containing substances of the general formula:

R<PF>-L<II>-G<III>,

whereas R<PF> represents a perfluoroalkyl group with 4 to 30 carbon atoms, L<II> stands for a linker and G<III> for a hydrophilic group. The linker L is a direct bond, an —SO2— group or a straight or branched carbon chain with up to 20 carbon atoms which can be substituted with one or more —OH, —COO<−>, —SO3-groups and/or, if necessary, one or more —O—, —S—, —CO—, —CONH—, —NHCO—, —CONR—, —NRCO—, —SO2-, —PO4-, —NH—, —NR-groups, an aryl ring or contain a piperazine, whereas R stands for a C1 to C20 alkyl group, which again can contain and/or have one or a plurality of O atoms and/or be substituted with —COO<−> or SO3- groups.

The hydrophilic group G<III> can be selected from a mono or disaccharide, one or a plurality of —COO<−> or —SO₃<−>-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO₂—(CH₂)₂—OH-group, a polyhydroxyalkyl chain with at least two hydroxyl groups or one or a plurality of polyethylene glycol chains having at least two glycol units, wherein the polyethylene glycol chains are terminated by an —OH or —OCH₃— group, or similar linkages.

In the exemplary embodiments of the present invention, paramagnetic metals in the form of metal complexes with phthalocyanines may be used to functionalize the implant, especially as described in Phthalocyanine Properties and Applications, Vol. 14, C. C. Leznoff and A. B. P. Lever, VCH Ed. Examples are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. 2004/214810.

Super-paramagnetic, ferromagnetic or ferrimagnetic signal-generating agents may also be used. For example, among magnetic metals, alloys are preferred, among ferrites such as gamma iron oxide, magnetites or cobalt-, nickel- or manganese-ferrites, corresponding agents are preferably selected, especially particles, as described in International Patent Publications WO83/03920, WO83/01738, WO85/02772 and WO89/03675, in U.S. Pat. Nos. 4,452,773 and 4,675,173, International Patent Publication WO88/00060 as well as U.S. Pat. No. 4,770,183, and International Patent Publications WO90/01295 and WO90/01899.

Further, magnetic, paramagnetic, diamagnetic or super paramagnetic metal oxide crystals having diameters of, e.g., less than about 4000 Angstroms are especially preferred as degradable non-organic diagnostic agents. Suitable metal oxides can be selected from iron oxide, cobalt oxides, iridium oxides or the like, which provide suitable signal producing properties and which have especially biocompatible properties or are biodegradable. Crystalline agents of such group having diameters smaller than 500 Angstroms may be used. These crystals can be associated covalently or non-covalently with macromolecular species. Further, zeolite-containing paramagnets and gadolinium-containing nanoparticles can be selected from polyoxometallates, preferably of the lanthanides (e.g., K9GdW10O36).

To optimize the image producing properties, the average particle size of the magnetic signal producing agents may be provided to 5 μm at maximum, such as from about 2 nm up to 1 μm, e.g. from about 5 nm to 200 nm. The super paramagnetic signal producing agents can be chosen, for example, from the group of so-called SPIOs (super paramagnetic iron oxides) with a particle size larger than 50 nm or from the group of the USPIOs (ultra small super paramagnetic iron oxides) with particle sizes smaller than 50 nm.

Signal-generating agents for imparting further functionality to the implants of embodiments of the present invention can further be selected from endohedral fullerenes, as described, for example, in U.S. Pat. No. 5,688,486 or International Patent Publication WO 93/15768, or from fullerene derivatives and their metal complexes such as fullerene species, which comprise carbon clusters having 60, 70, 76, 78, 82, 84, 90, 96 or more carbon atoms. An overview of such species can be gathered from European Patent Application 1331226A2. Metal fullerenes or endohedral carbon-carbon nanoparticles with arbitrary metal-based components can also be selected. Such endohedral fullerenes or endometallo fullerenes may contain, for example, rare earths such as cerium, neodymium, samarium, europium, gadolinium, terbium, dysprosium or holmium. The choice of nanomorphous carbon species is not limited to fullerenes; other nanomorphous carbon species such as nanotubes, onions, etc. may also be applicable.

In another exemplary embodiment, fullerene species may be selected from non-endohedral or endohedral forms which contain halogenated, preferably iodated, groups, as described in U.S. Pat. No. 6,660,248.

Generally, mixtures of such signal-generating agents of different specifications can also used, depending on the desired properties of the signal-generating material properties. The signal producing agents used can have a size of 0.5 nm to 1,000 nm, preferably 0.5 nm to 900 nm, especially preferred from 0.7 to 100 nm, and may partly replace the metal-based particles. Nanoparticles are easily modifiable based on their large surface to volume ratios. The nanoparticles can, for example, be modified non-covalently by means of hydrophobic ligands, for example with trioctylphosphine, or be covalently modified. Examples of covalent ligands are thiol fatty acids, amino fatty acids, fatty acid alcohols, fatty acids, fatty acid ester groups or mixtures thereof, for example oleic acid and oleylamine.

In the exemplary embodiments of the present invention, the active ingredients such as signal producing agents can be encapsulated in micelles or liposomes with the use of amphiphilic components, or may be encapsulated in polymeric shells, wherein the micelles/liposomes can have a diameter of 2 nm to 800 nm, preferably from 5 to 200 nm, especially preferred from 10 to 25 nm. The micelles/liposomes may be added to the suspension before molding, to be incorporated into the implant. The size of the micelles/liposomes is, without committing to a specific theory, dependant on the number of hydrophobic and hydrophilic groups, the molecular weight of the nanoparticles and the aggregation number. In aqueous solutions the use of branched or unbranched amphiphilic substances, is especially preferred in order to achieve the encapsulation of signal-generating agents in liposomes/micelles. The hydrophobic nucleus of the micelles hereby contains in an exemplary embodiment a multiplicity of hydrophobic groups, preferably between 1 and 200, especially preferred between 1 and 100 and mostly preferred between 1 and 30 according to the desired setting of the micelle size.

Such signal-generating agents encapsulated in micelles and incorporated into the porous implant can, moreover, be functionalized, while linker (groups) are attached at any desired position, preferably amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound according to the prior art. Here, especially biological molecules such as proteins, peptides, amino acids, polypeptides, lipoproteins, glycosaminoglycanes, DNA, RNA or similar biomolecules are preferred especially.

Signal-generating agents may also be selected from non-metal-based signal generating agents, for example from the group of X-ray contrast agents, which can be ionic or non-ionic. Among the ionic contrast agents are included salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy 1]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3-[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1(2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like especially preferred, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. No. 2,705,726, U.S. Pat. No. 2,895,988, Chem. Ber. 93:2347 (1960), SA-A-68/01614, Acta Radiol. 12: 882 (1972), British Patent 870321, Rec. Trav. Chim. 87: 308 (1968), East German Patent 67209, German Patent 2050217, German Patent 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent 1346796, U.S. Pat. No. 2,551,696, U.S. Pat. No. 1,993,039, Ann 494: 284 (1932), J. Pharm. Soc. (Japan) 50: 727 (1930), and U.S. Pat. No. 4,005,188.

Examples of applicable non-ionic X-ray contrast agents in accordance with the exemplary embodiments of the present invention are metrizamide as disclosed in German Application DE-A-2031724, iopamidol as disclosed in BE-A-836355, iohexyl as disclosed in British Application GB-A-1548594, iotrolan as disclosed in European Application EP-A-33426, iodecimol as disclosed in European Application EP-A-49745, iodixanol as in European Application EP-A-108638, ioglucol as disclosed in U.S. Pat. No. 4,314,055, ioglucomide as disclosed in BE-A-846657, ioglunioe as disclosed in German Application DE-A-2456685, iogulamide as in BE-A-882309, iomeprol as disclosed in European Application EP-A-26281, iopentol as disclosed in European Application EP-A-105752, iopromide as disclosed in German Application DE-A-2909439, iosarcol as disclosed in German Application DE-A-3407473, iosimide as disclosed in German Application DE-A-3001292, iotasul as disclosed in European Application EP-A-22056, iovarsul as disclosed in European Application EP-A-83964 or ioxilan as disclosed in International Publication WO87/00757.

Agents based on nanoparticle signal-generating agents may be selected to impart functionality to the implant, which after release into tissues and cells are incorporated or are enriched in intermediate cell compartments and/or have an especially long residence time in the organism.

Such particles can include water-insoluble agents, a heavy element such as iodine or barium, PH-50 as monomer, oligomer or polymer (iodinated aroyloxy ester having the empirical formula C19H2313N2O6, and the chemical names 6-ethoxy-6-oxohexy-3,5-bis(acetyl amino)-2,4,6-triiodobenzoate), an ester of diatrizoic acid, an iodinated aroyloxy ester, or combinations thereof. Particle sizes which can be incorporated by macrophages may be preferred. A corresponding method for this is disclosed in International Patent Publication WO03/039601 and suitable agents are described in U.S. Pat. Nos. 5,322,679, 5,466,440, 5,518,187, 5,580,579, and 5,718,388. Nanoparticles which are marked with signal-generating agents or such signal generating agents such as PH-50, which accumulate in intercellular spaces and can make interstitial as well as extrastitial compartments visible, can be advantageous.

Signal-generating agents may also include anionic or cationic lipids, as disclosed in U.S. Pat. No. 6,808,720, for example, anionic lipids such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, etc. Moreover, halogenated, in particular fluorinated anionic lipids can be preferred in exemplary embodiments. The anionic lipids preferably contain cations from the alkaline earth metals beryllium (Be<+2>), magnesium (Mg<+2>), calcium (Ca<+2>), strontium (Sr<+2>) and barium (Ba<+2>), or amphoteric ions, such as aluminum (Al<+3>), gallium (Ga<+3>), germanium (Ge<+3>), tin (Sn+<4>) or lead (Pb<+2> and Pb<+4>), or transition metals such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> und Ir<+4>), mercury (Hg<+2>) or bismuth (Bi<+3>), and/or rare earths such as lanthanides, for example lanthanum (La<+3>) and gadolinium (Gd<+3>). Cations can include calcium (Ca<+2>), magnesium (Mg<+2>) and zinc (Zn<+2>) and paramagnetic cations such as manganese (Mn<+2>) or gadolinium (Gd<+3>).

Cationic lipids may include phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters, and also derivatives such as N-[1-(2,3-dioleoyloxy)propyl]-N,N,N-trimethylammonium chloride (“DOTMA”); furthermore, synthetic cationic lipids based on, for example, naturally occurring lipids such as dimethyldioctadecylammonium bromide, sphingolipids, sphingomyelin, lysolipids, glycolipids such as, for example, gangliosides GM1, sulfatides, glycosphingolipids, cholesterol und cholesterol esters or salts, N-succinyldioleoylphosphatidyl ethanolamine, 1,2,-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, and fluorinated, derivatized cationic lipids, as disclosed in U.S. Ser. No. 08/391,938. Such lipids are furthermore suitable as components of signal-generating liposomes, which especially can have pH-sensitive properties as disclosed in U.S. 2004197392 and incorporated herein explicitly.

Signal-generating agents may also include so-called micro bubbles or micro balloons, which contain stable dispersions or suspensions in a liquid carrier substance. Suitable gases may include air, nitrogen, carbon dioxide, hydrogen or noble gases such as helium, argon, xenon or krypton, or sulfur-containing fluorinated gases such as sulfur hexafluoride, disulfurdecafluoride or trifluoromethylsulfurpentafluoride, or for example selenium hexafluoride, or halogenated silanes such as methylsilane or dimethylsilane, further short chain hydrocarbons such as alkanes, specifically methane, ethane, propane, butane or pentane, or cycloalkanes such as cyclopropane, cyclobutane or cyclopentane, also alkenes such as ethylene, propene, propadiene or butene, or also alkynes such as acetylene or propyne. Further ethers such as dimethylether may be selected, or ketones, or esters or halogenated short-chain hydrocarbons or any desired mixtures of the above. Examples further include halogenated or fluorinated hydrocarbon gases such as bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane or perfluorohydrocarbons such as, for example, perfluoroalkanes, perfluorocycloalkanes, perfluoroalkenes or perfluorinated alkynes. Especially preferred are emulsions of liquid dodecafluoropentane or decafluorobutane and sorbitol, or similar, as disclosed in WO-A-93/05819.

Preferably, such micro bubbles are selected, which are encapsulated in compounds having the structure
R1-X-Z;
R2-X-Z; or
R3-X-Z′
   whereas R1, R2 and R3 comprise hydrophobic groups selected from straight chain alkylenes, alkyl ethers, alkyl thiol ethers, alkyl disulfides, polyfluoroalkylenes and polyfluoroalkylethers, Z comprises a polar group from CO2-M<+>, SO3<−> M<+>, SO4<−> M<+>, PO3<−> M<+>, PO4<−> M<+>2, N(R)4<+> or a pyridine or substituted pyridine, and a zwitterionic group, and finally X represents a linker which binds the polar group with the residues.

Gas-filled or in situ out-gassing micro spheres having a size of <1000 μm can be further selected from biocompatible synthetic polymers or copolymers which comprise monomers, dimers or oligomers or other pre-polymer to pre-stages of the following polymerizable substances: acrylic acid, methacrylic acid, ethyleneimine, crotonic acid, acryl amide, ethyl acrylate, methylmethacrylate, 2-hydroxyethylmethacrylate (HEMA), lactonic acid, glycolic acid, [epsilon]caprolactone, acrolein, cyanoacrylate, bisphenol A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamides, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium-2-sulfoxyethylmethacrylate, vinyl pyridine, aminoethylmethacrylate, 2-methacryloyloxytrimethylammonium chloride, and polyvinylidenes, such as polyfunctional cross-linkable monomers such as, for example, N,N′-methylene-bis-acrylamide, ethylene glycol dimethacrylate, 2,2′-(p-phenylenedioxy)-diethyldimethacrylate, divinylbenzene, triallylamine and methylene-bis-(4-phenyl-isocyanate), including any desired combinations thereof. Preferred polymers contain polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamides (e.g. Nylon) and the like, or any arbitrary mixtures thereof. Preferred copolymers contain among others polyvinylidene-polyacrylonitrile, polyvinylidene-polyacrylonitrile-polymethylmethacrylate, and polystyrene-polyacrylonitrile and the like, or any desired mixtures thereof. Methods for manufacture of such micro spheres are published, for example, in Garner et al., U.S. Pat. No. 4,179,546, Garner, U.S. Pat. No. 3,945,956, Cohrs et al., U.S. Pat. No. 4,108,806, Japan Kokai Tokkyo Koho Publication 62 286534, British Patent No. 1,044,680, Kenaga et al., U.S. Pat. No. 3,293,114, Morehouse et al., U.S. Pat. No. 3,401,475, Walters, U.S. Pat. No. 3,479,811, Walters et al., U.S. Pat. No. 3,488,714, Morehouse et al., U.S. Pat. No. 3,615,972, Baker et al., U.S. Pat. No. 4,549,892, Sands et al., U.S. Pat. No. 4,540,629, Sands et al., U.S. Pat. No. 4,421,562, Sands, U.S. Pat. No. 4,420,442, Mathiowitz et al., U.S. Pat. No. 4,898,734, Lencki et al., U.S. Pat. No. 4,822,534, Herbig et al., U.S. Pat. No. 3,732,172, Himmel et al., U.S. Pat. No. 3,594,326, Sommerville et al., U.S. Pat. No. 3,015,128, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964).

Other signal-generating agents can be selected from agents which may be transformed into signal generating agents in organisms by means of in-vitro or in-vivo cells, cells as a component of cell cultures, of in-vitro tissues, or cells as a component of multicellular organisms, such as, for example, fungi, plants or animals, in exemplary embodiments from mammals such as mice or humans. Such agents can be made available in the form of vectors for the transfection of multicellular organisms, wherein the vectors contain recombinant nucleic acids for the coding of signal-generating agents. In exemplary embodiments, this may be done with signal-generating agents such as metal binding proteins. It can be preferred to choose such vectors from the group of viruses, for example, from adeno viruses, adeno virus associated viruses, herpes simplex viruses, retroviruses, alpha viruses, pox viruses, arena-viruses, vaccinia viruses, influenza viruses, polio viruses or hybrids of any of the above.

Such signal-generating agents may be used in combination with exemplary delivery systems, e.g., in order to incorporate nucleic acids, which are suitable for coding for signal-generating agents, into the target structure. Virus particles for the transfection of mammalian cells may be used, wherein the virus particle contains one or a plurality of coding sequence/s for one or a plurality of signal generating agents as described above. In these cases, the particles can be generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

These signal-generating agents can be made available from colloidal suspensions or emulsions, which are suitable to transfect cells, preferably mammalian cells, wherein these colloidal suspensions and emulsions contain those nucleic acids which possess one or a plurality of the coding sequence(s) for signal generating agents. Such colloidal suspensions or emulsions can include macromolecular complexes, nano capsules, micro spheres, beads, micelles, oil-in-water- or water-in-oil emulsions, mixed micelles and liposomes or any desired mixture of the above.

In addition, cells, cell cultures, organized cell cultures, tissues, organs of desired species and non-human organisms can be chosen which contain recombinant nucleic acids having coding sequences for signal generating agents. In exemplary embodiments organisms can include mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal-generating agents can be produced in vivo from proteins and made available as described above. Such agents can be directly or indirectly signal producing, while the cells produce (direct) a signal producing protein through transfection, or produce a protein which induces (indirect) the production of a signal producing protein. These signal generating agents are e.g. detectable in methods such as MRI, while the relaxation times T1, T2, or both are altered and lead to signal producing effects which can be processed sufficiently for imaging. Such proteins can include protein complexes, such as metalloprotein complexes. Direct signal producing proteins can include such metalloprotein complexes which are formed in the cells. Indirect signal producing agents can include proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), transferrin receptor (for the take-up of Fe), erythroid-5-aminobevulinate synthase (for the utilization of Fe, H-Ferritin and L-Ferritin for the purpose of Fe storage). In exemplary embodiments, both types of signal-generating agents, that is direct and indirect, may be combined with each other, for example an indirect signal-generating agent, which regulates the iron-homeostasis and a direct agent, which represents a metal-binding protein.

In the exemplary embodiments of the present invention where metal-binding polypeptides are selected as indirect agents, it can be advantageous if the polypeptide binds to one or a plurality of metals which possess signal generating properties. Metals with unpaired electrons in the Dorf orbitals may be used, such as, for example, Fe, Co, Mn, Ni, Gd etc., whereas especially Fe is available in high physiological concentrations in organisms. Such agents may form metal-rich aggregates, for example crystalline aggregates, whose diameters are larger than 10 picometers, preferably larger than 100 picometers, 1 nm, 10 nm or specially preferred larger than 100 nm.

In addition, metal-binding compounds which have sub-nanomolar affinities with dissociation constants of less than 10-15 M, 10-2 M or smaller may be used to impart functionality for the implant. Typical polypeptides or metal-binding proteins are lactoferrin, ferritin, or other dimetallocarboxylate proteins, or so-called metal catchers with siderophoric groups, such as hemoglobin. A possible exemplary method for preparation of such signal generating agents, their selection and the possible direct or indirect agents which are producible in vivo and are suitable as signal generating agents is described in International Patent Publication WO 03/075747.

Another group of signal-generating agents can be photo physically signal producing agents which consist of dyestuff-peptide-conjugates. Such dyestuff-peptide-conjugates can provide a wide spectrum of absorption maxima, for example polymethin dyestuffs, such as cyanine-, merocyanine-, oxonol- and squarilium dyestuffs. From the class of the polymethin dyestuffs, the cyanine dyestuffs, e.g. the indole structure based indocarbo-, indodicarbo- and indotricarbocyanines, can be suitable. Such dyestuffs can be substituted with suitable linking agents and can be functionalized with other groups as desired, see also German Application DE 19917713.

The signal-generating agents can further be functionalized as desired. The functionalization by means of so-called “Targeting” groups is meant to include functional chemical compounds which link the signal generating agent or its specifically available form (encapsulation, micelles, micro spheres, vectors etc.) to a specific functional location, or to a determined cell type, tissue type or other desired target structures. Targeting groups can permit the accumulation of signal-producing agents in or at specific target structures. Therefore, the targeting groups can be selected from such substances, which are principally suitable to provide a purposeful enrichment of the signal generating agents in their specifically available form by physical, chemical or biological routes or combinations thereof. Useful targeting groups can, therefore, include antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. Exemplary targeting groups may include those which enrich signal-generating agents in/on a tissue type or on surfaces of cells. Here it may not be necessary for the function that the signal-generating agent is taken up into the cytoplasm of the cells. Peptides can be targeting groups, for example chemotactic peptides that are used to visualize inflammation reactions in tissues by means of signal-generating agents; see also Internation Publication No. WO 97/14443.

Antibodies can be used, including antibody fragments, Fab, Fab2, Single Chain Antibodies (for example Fv), chimerical antibodies, moreover antibody-like substances, for example so-called anticalines, wherein it may not be important whether the antibodies are modified after preparation, recombinants are produced or whether they are human or non-human antibodies. Humanized or human antibodies may be used, such as chimerical immunoglobulines, immunoglobulin chains or fragments (such as Fv, Fab, Fab′, F(ab″)2 or other antigen-binding subsequences of antibodies, which may partly contain sequences of non-human antibodies; humanized antibodies may include human immunoglobulines (receptor or recipient antibody), in which groups of a CDR (Complementary Determining Region) of the receptor are replaced through groups of a CDR of a non-human (spender or donor antibody), wherein the spender species for example, mouse, rabbit or other has appropriate specificity, affinity, and capacity for the binding of target antigens. In a few forms the Fv framework groups of the human immunglobulines are replaced by means of corresponding non-human groups. Humanized antibodies can, moreover, contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. Targeting groups can also include hetero-conjugated antibodies. The functions of the selected antibodies or peptides include cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc.

Moreover, targeting groups may contain the functional binding sites of ligands which are suitable for binding to any desired cell receptors. Examples of target receptors include receptors of the group of insulin receptors, insulin-such as growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, estrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

Further, hormone receptors may be used, especially for hormones such as steroidal hormones or protein- or peptide-based hormones, for example, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cx(H2O)_{y}, wherein herewith also monosaccharides, disaccharides and oligo- as well as polysaccharides are included, as well as other polymers which consist of sugar molecules which contain glycosidic bonds. Carbohydrates may include those in which all or parts of the carbohydrate components contain glycosylated proteins, including the monomers and oligomers of galactose, mannose, fructose, galactosamine, glucosamine, glucose, sialic acid, and the glycosylated components, which make possible the binding to specific receptors, especially cell surface receptors. Other useful carbohydrates include monomers and polymers of glucose, ribose, lactose, raffinose, fructose and other biologically occurring carbohydrates especially polysaccharides, for example, arabinogalactan, gum Arabica, mannan etc., which are suitable for introducing signal generating agents into cells, see U.S. Pat. No. 5,554,386.

Furthermore, targeting groups can include lipids, fats, fatty oils, waxes, phospholipids, glycolipids, terpenes, fatty acids and glycerides, and triglycerides, or eicosanoides, steroids, sterols, suitable compounds of which can also be hormones such as prostaglandins, opiates and cholesterol etc. All functional groups can be selected as the targeting group, which possess inhibiting properties, such as, for example, enzyme inhibitors, preferably those which link signal generating agents into/onto enzymes.

Targeting groups can also include functional compounds which enable internalization or incorporation of signal generating agents in the cells, especially in the cytoplasm or in specific cell compartments or organelles, such as for example the cell nucleus. For example, such a targeting group may contains all or parts of HIV-1 tat-proteins, their analogues and derivatized or functionally similar proteins, and in this way allows an especially rapid uptake of substances into the cells. As an example refer to Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990).

Targeting groups can further include the so-called Nuclear Localisation Signal (NLS), which include positively charged (basic) domains which bind to specifically targeted structures of cell nuclei. Numerous NLS and their amino acid sequences are known including single basic NLS such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see for example Boulikas, J. Cell. Biochem. 55(1):32-58 (1994), and double basic NLS's such as, for example, xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. Numerous localization studies have shown that NLSs, which are built into synthetic peptides which normally do not address the cell nucleus or were coupled to reporter proteins, lead to an enrichment of such proteins and peptides in cell nuclei. Exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. Targeting groups for the hepatobiliary system may be selected, as suggested in U.S. Pat. Nos. 5,573,752 and 5,582,814.

In the exemplary embodiments of the present invention, the implant comprises absorptive agents, e.g. to remove compounds from body fluids. Suitable absorptive agents include chelating agents such as penicillamine, methylene tetramine dihydrochloride, EDTA, DMSA or deferoxamine mesylate, any other appropriate chemical modification, antibodies, and micro beads or other materials containing cross linked reagents for absorption of drugs, toxins or other agents.

According to the exemplary embodiments of the present invention, functional modification can be achieved by incorporating at least one beneficial agent as defined herein partially or completely into or onto the implant structure. Incorporation may be carried out by any suitable means, such as impregnating, dip-coating, spray coating or the like. The beneficial agent diagnostic agent or absorptive agent may be provided in an appropriate solvent, optionally using additives. The loading of these agents may be carried out under atmospheric, sub-atmospheric pressure or under vacuum. Alternatively, loading may be carried out under high pressure. Incorporation of the beneficial agent may be carried out by applying electrical charge to the implant or exposing at least a portion of the implant to a gaseous material including the gaseous or vapor phase of the solvent, in which an agent is dissolved or other gases that have a high degree of solubility in the loading solvent. In further exemplary embodiments, the beneficial agents like biologically, pharmacologically, therapeutically active agents, diagnostic agents or absorptive agents are provided in the polymer particles which serve as a carrier therefore, and which are embedded in the matrix of the metal-based particles of the implant.

Functional modification can also be achieved by selecting the particles appropriately with regard to their biochemical, physical and biological properties. One exemplary embodiment can include the use of x-ray absorptive particles such as tantalum, tungsten etc. as at least a part of the metal based particles. In other exemplary embodiments ferromagnetic metal-based particles may be used to achieve visibility in MRI imaging.

Functional modification can also be implemented by adding a beneficial agent, such as a biologically, pharmacologically, therapeutically active agents, diagnostic and/or absorptive agents partially or completely to the surface of the inventive implant, for example in a coating.

In other exemplary embodiments of the present invention, the beneficial agents, as defined herein can be added by introducing them encapsulated, preferably encapsulated in polymeric shells, into the implant body. In these exemplary embodiments, the agents can represent the polymer particles and the encapsulating material is selected from materials as defined above for the biodegradable polymer particles that allow eluting of the active ingredients by partially or completely dissolving the encapsulating material in physiologic fluids.

Further functional modification can be achieved by adding, partially or completely incorporating a material that alters and modulates, hereinafter referred to as altering and modulating material, the availability, function or release of a therapeutically active agent, diagnostic and/or absorptive agents. The altering and modulating material may comprise a diffusion barrier or a biodegradable material or a polymer or hydrogel. In some exemplary embodiments, the biodegradable polymer particles may further comprise a combination of different beneficial agents as defined herein that are incorporated into different altering and modulating materials.

In other exemplary embodiments of the present invention, functional modification can be carried out by application of a coating of one or more altering and modulating materials onto at least one part of the implant, whereby the polymer particles of the device comprise at least one therapeutically active agent, diagnostic or absorptive agent.

In other exemplary embodiments of the present invention, it can be of advantage to coat the implant, or at least a part of the implant, with non-degradable or degradable polymers, optionally containing therapeutically, or diagnostically or absorptive agents or any mixture thereof.

In another exemplary embodiment of the present invention, it can be desirable to coat the implant on the outer surface or inner surface with a coating to enhance engraftment or biocompatibility. Such coatings may comprise carbon coatings, metal carbides, metal nitrides, metal oxides e.g. diamond-like carbon or silicon carbide, or pure metal layers of e.g. titanium, using PVD, Sputter-, CVD or similar vapor deposition methods or ion implantation.

In further exemplary embodiments of the present invention, it may be preferred to produce a porous coating onto at least one part of the inventive implant in a further step, such as porous carbon coatings, as described in International Patent Publication WO 2004/101177, WO 2004/101017 or WO 2004/105826, or porous composite-coatings, as described previously in International Patent Application PCT/EP2006/063450, or porous metal-based coatings, as disclosed in International Patent Publication WO 2006/097503, or any other suitable porous coating.

In further exemplary embodiments of the present invention, a sol/gel-based beneficial agent can be incorporated into the inventive implant or a sol/gel-based coating that can be dissolvable in physiological fluids may be applied to at least a part of the implant, as disclosed, e.g., in International Patent Publication WO 2006/077256 or WO 2006/082221.

In some exemplary embodiments of the present invention, it can be desirable to combine two or more different functional modifications as described above to obtain a functional implant.

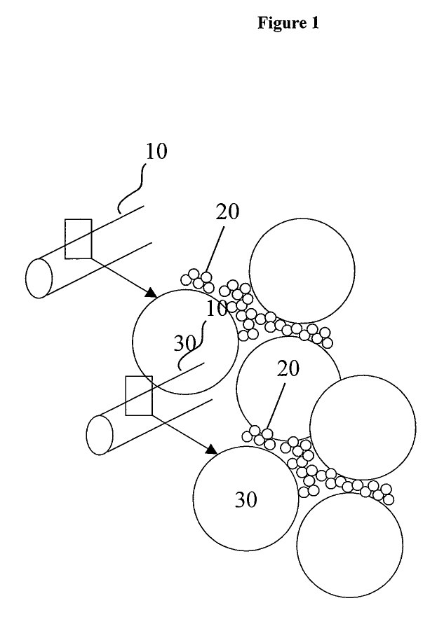

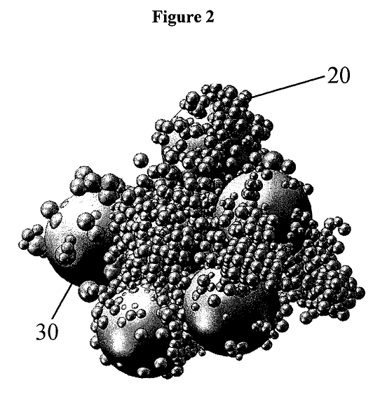

FIG. 1 shows a schematic illustration, at the left hand side a tubular implant (10), of an exemplary embodiment of the present invention, and a partial magnification of the structure thereof illustrating a structure that is composed of or manufactured from a plurality of spherical particles (20) surrounding larger voids (30) left over from removed particles. FIG. 2 shows an illustration of a three-dimensional orientation of the spherical particles (20) surrounding larger voids (30) left over from removed particles.

### EXAMPLES

### Example 1

A slurry was produced using Mg nanoparticles and polyethylene beads. Mg nanoparticles was purchased from Metal Nanopowders Limited and polyethylene beads from Impag. The slurry was produced using 200 g of Mg nanoparticles (particle size D50 of about 50 nm) by adding 100 g acetone, stirring its for approximately 1 hour and adding 150 g of polyethylene beads. The slurry was homogenized for another 90 minutes.

### Example 2

### Molding of Discoid Implants

### Rapid Heating

A standard cylindrical hollow mold made out of stainless steel was used with an inner diameter of 3 cm and a length of 8 cm. The slurry of example 1 was filled into the mold until ⅘ of the volume was filled and compacting was carried out by using a standard floating mold die press to form a green body. Subsequently, a compaction pressure of 20 MPa was applied for 40 seconds, then repeating the cycle two further times. The green body comprised a discoid type mold with a diameter of 2.8 cm and a height of 2.5 cm. It was further dried at room temperature for 1 hour and then put into a standard tube reactor. The green body was sintered with a heating ramp of 20 K/min at 600° C. for 4 hours and then cooled down to room temperature within 20 hours. The thermal treatment was carried out under a nitrogen atmosphere at a N₂-flow rate of 1000 ml/min.

The molded body was cut to analyse the pore structure induced by the polyethylene bead filler. The molded body showed macroscopically a regular surface structure. The fine structure was analyzed using field emission scanning microscopy (FESEM). The fine structure of the molded body showed a net shape imprint of the polythelene particles.

### Example 3

### Molding of Discoid Implants

### Two Step Heat Treatment (Comparative Example)

The process of compacting was repeated according to example 2 with slurry of example 1 within the same mold. The green body comprised a discoid type mold with a diameter of 2.9 cm and a height of 2.6 cm. It was further dried at room temperature for 1 hour and then put into a standard tube reactor. The green body was thermally treated in two steps, first applying a heating ramp of 2 K/min up to 120° C., keeping 120° C. for approximately 1 hour, and then with the same ramp of 2K/min to 600° C. for 4 hours and then cooled down to room temperature within 20 hours. The thermal treatment was carried out under a nitrogen atmosphere at a N₂-flow rate of 1000 ml/min.

The molded body was cut to analyze the pore structure induced by the polyethylene bead filler. The molded body showed macroscopically a irregular surface structure. The fine structure was analyzed using FESEM. The FESEM image showed that the net shape was not regular and the fine structure was significantly destroyed, the average pore size compared to the material obtained in example 2 above was 10 times lower, indicating a collapse of the larger pores.

### Example 4

### Molding of Discoid Implants

### Two Step Heat Treatment (Comparative Example)

The process of compacting was repeated according to example 2 with slurry of example 1 within the same mold. The green body comprised a discoid type mold with a diameter of 2.9 cm and a height of 2.8 cm. It was further dried at room temperature for 1 hour and then put into a standard tube reactor. The green body was thermally treated in two steps, first applying a heating ramp of 20 K/min up to 120° C., keeping 120° C. for approximately 1 hour, and then with the same ramp of 20K/min to 600° C. for 4 hours and then cooled down to room temperature within 20 hours. The thermal treatment was carried out under a nitrogen atmosphere at a N₂-flow rate of 1000 ml/min.

The molded body was cut to analyse the pore structure induced by the polyethylene bead filler. The molded body showed macroscopically a irregular surface structure. The fine structure was analyzed using FESEM. The FESEM image showed that the net shape was not regular and the fine structure was significantly destroyed, the average pore size compared to the material obtained in example 2 above was 15 times lower, indicating collapse of the larger pores.

Having thus described in detail several exemplary embodiments of the present invention, it is to be understood that the invention described above is not to be limited to particular details set forth in the above description, as many apparent variations thereof are possible without departing from the spirit or scope of the present invention. The embodiments of the present invention are disclosed herein or are obvious from and encompassed by the detailed description. The detailed description, given by way of example, but not intended to limit the invention solely to the specific embodiments described, may best be understood in conjunction with the accompanying Figures.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are entirely hereby incorporated herein by reference, and may be employed in the practice of the invention. Citation or identification of any document in this application is not an admission that such document is available as prior art to the present invention.

1. A method for producing an at least partially biodegradable porous implant or a part thereof, comprising:
providing a suspension comprising a plurality of first particles of at least one organic polymer, a plurality of second particles of at least one metal-based material which is at least partially biodegradable in-vivo, and at least one solvent, wherein the first and second particles are substantially insoluble in the at least one solvent;
molding the suspension to form a green body comprising the first particles embedded in a matrix of the second particles which are compressed;
removing the first particles from the green body by at least one of thermally-induced decomposition or evaporation; and
sintering the green body to form the implant or the part thereof, the first particles are removed during the sintering of the green body.

2. The method of claim 1, wherein the suspension is molded by one of compacting, injection molding, uniaxial or biaxial pressing, isostatic pressing, slip casting, or extrusion molding.

3. The method of claim 1, wherein the suspension comprises the first and second particles in a volume ratio from about 30:1 to 1:30.

4. The method of claim 1, wherein the combined weight of the first and second particles in the suspension amount to more than about 50 wt-% of the suspension in total.

5. The method of claim 1, wherein the suspension has paste-like properties.

6. The method of claim 1, wherein the suspension comprises at least one further additive selected from dispersants or surfactants.

7. The method of claim 1, wherein the molding procedure uses compaction pressures in the range of from about 6,890 kPa (1,000 psi) to about 138,000 kPa (20,000 psi).

8. The method of claim 1, wherein the molding procedures uses compaction times in the range of from about 1 second to about 6000 seconds.

9. The method of claim 1, wherein the suspension is molded by an injection molding.

10. The method of claim 1, wherein the first and second particles are independently selected from at least one of spherical particles, dendritic particles, cubes, wires, fibers or tubes.

11. The method of claim 1, wherein the second metal-based particles include at least one of a metal, a metal alloy, a metal oxide, a metal carbide, a metal nitride, or a metal containing semiconductor.

12. The method of claim 11, wherein the metal or metal alloy is selected from Mg or Zn, or an alloy comprising at least one of Mg, Ca, Fe, Zn, Al, W, Ln, Si, or Y.

13. The method of claim 11, wherein the second particles comprise a combination of metal or metal alloy particles selected from Mg or an alloy comprising at least one of Mg, Ca, Fe, Zn, Al, W, Ln, Si, or Y, and particles selected from at least one of Mn, Co, Ni, Cr, Cu, Cd, Pb, Sn, Th, Zr, Ag, Au, Pd, Pt, Si, Ca, Li, Al, Zn, or Fe.

14. The method of claim 11, wherein the second particles comprise
(i) 10-98 wt.-% of Mg, and 0-70 wt.-% Li and 0-12 wt.-% of other metals, or
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals.

15. The method of claim 11, wherein the first and second particles, independently of each other, have an average particle size in the range from about 0.5 nanometers to 500 micrometers.

16. The method of claim 15, wherein the average particle size of the first particles is greater than the average particle size of the second particles.

17. The method of claim 1, wherein the first particles are removed by continuously heating the green body with a heating ramps of from about 5 K/min up to 20 K/min, substantially without interruption or plateaus in the temperature profile up to reaching the final sintering temperature.

18. The method of claim 17, wherein the geren body is heated at the heating ramps from about 15 to 25 K/min, and at most about 20 K/min to the final sintering temperature

19. At least partially biodegradable porous implant, comprising:
at least one portion which is produced by the procedures comprising:
providing a suspension comprising a plurality of first particles of at least one organic polymer, a plurality of second particles of at least one metal-based material which is at least partially biodegradable in-vivo, and at least one solvent, wherein the first and second particles are substantially insoluble in the at least one solvent,
molding the suspension to form a green body comprising the first particles embedded in a matrix of the second particles which are compressed,
removing the first particles from the green body by at least one of thermally-induced decomposition or evaporation, and
sintering the green body to form the implant or the part thereof, the first particles are removed during the sintering of the green body.

20. The implant of claim 19, wherein the at least one portion comprises at least one active ingredient.

21. The implant of claim 20, wherein the at least one active ingredient is configured to be released in-vivo.

22. The implant of claim 19, wherein the active ingredient includes at least one of a pharmacologically, therapeutically, biologically or diagnostically active agent or an absorptive agent.

23. The implant of claim 20, wherein the second particles include at least one of a therapeutically active agent or a diagnostically active agent.

22. The implant of claim 20, wherein the implant is selected from the group consisting of a vascular endoprosthesis, an intraluminal endoprosthesis, a stent, a stent graft, a coronary stent, a peripheral stent, a surgical or orthopedic or dental implant, an implantable orthopedic fixation aid, an orthopedic bone prosthesis or joint prosthesis, a bone substitute or a vertebral substitute in the thoracic or lumbar region of the spinal column; an artificial heart or a part thereof, an artificial heart valve, a heart pacemaker casing or electrode, a subcutaneous or intramuscular implant, an implantable drug-delivery device, a microchip, or implantable surgical needles, screws, nails, clips, staples, or seed implants.
